(19) United States
(12) Patent Application Publication
(10) Pub. No.: US 2021/0259749 A1
(43) Pub. Date: Aug. 26, 2021

(54) A DEVICE AND METHOD FOR FIXATION OF BONE FRAGMENTS IN AN OSTEOTOMY PROCEDURE

(71) Applicant: Cab Medical Pty Ltd, Sydney (AU)

(72) Inventors: Peter Lam, Chatswood (AU); Bradley Ryan, Sydney (AU)

(21) Appl. No.: 17/271,878

(22) PCT Filed: Aug. 30, 2019
(86) PCT No.: PCT/AU2019/050924

§ 371 Date: Feb. 26, 2021

(30) Foreign Application Priority Data
Aug. 31, 2018 (AU) 2018903230

(51) Int. Cl. A61B 17/72

(52) U.S. Cl. CPC A61B 17/725; A61B 2017/565; A61B 17/7291

(57) ABSTRACT

The present disclosure relates to a device and method for the fixation of bone fragments in an osteotomy procedure. The device includes a first screw including a shank and an external thread for cortical fixation in the bone body fragment and an opening extending transversely through the shank. The device also includes a second screw including a shank and an external thread for cortical fixation in the bone head fragment for relative fixation of the bone body fragment and the bone head fragment. The shank of the second screw being locatable within the transverse opening of the first screw. The disclosure also relates to a jig mountable to the first screw and including one or more guides to align with the one or more transverse openings through the first screw.

### CROSS REFERENCE TO RELATED APPLICATIONS

This application is a U.S. national stage application under 35 U.S.C. 371 of PCT Application No. PCT/AU2019/050924, filed Aug. 30, 2019, which claims the benefit of AU 2018903230, filed Aug. 31, 2018, the disclosures of which are hereby incorporated by reference in their entirety.

### TECHNICAL FIELD

The present disclosure relates to a device and method for the fixation of bone fragments in an osteotomy procedure. The device and method are particularly suitable for use in first metatarsal osteotomy for the correction of hallux valgus deformity although it is to be appreciated that the present invention may have broader application.

### BACKGROUND

Hallux Valgus, commonly referred to as “bunions”, is a condition where there is a medial deviation of the first metatarsal and lateral deviation of the great toe or hallux. “Valgus” refers to the abnormal slant of the great toe. “Bunion” refers to the pathological bump or inflammation on the side of the great toe joint between the first metatarsal and the proximal phalanx associated with either a bursal sac or a bony deformity involving the first metatarsal bone.

Bunion development can be caused by a biomechanical abnormality, where certain tendons, ligaments, and supportive structures of the first metatarsal are no longer functioning correctly. This biomechanical abnormality may be due to the structure of the foot (e.g. flat feet), excessive ligamentous flexibility and abnormal bone structure.

The appropriate treatment of hallux valgus deformity will depend on an assessment of the hallux valgus angle (HVA), the intermetatarsal angle (IMA) and the contribution of distal metatarsophalangeal joint congruity.

Surgical procedures may address some combination of removing the abnormal bony enlargement of the first metatarsal, realigning the first metatarsal relative to the adjacent metatarsal, straightening the hallux or great toe relative to the first metatarsal and adjacent toes, realigning the cartilagenous surfaces of the great toe joint, repositioning the sesamoid bones beneath the first metatarsal and correcting any abnormal bowing or misalignment within the great toe.

Various methods to correct the intermetatarsal angle are known. When more rigid deformities of the intermetatarsal angle are present, it is generally reduced by using a distal or proximal osteotomy of the first metatarsal. This is where a surgeon cuts into the foot near the bunion and removes the excess growth of bone with a bone saw. Depending on the degree of deformity, the surgeon may need to cut into the bone of the great toe and realign the bones so that the great toe no longer slants to the outside. Improving the angle of the great toe and repairing the metatarsal bones may require an implant to fix them in place. The incisions are later closed with stitches, and a bandage is applied.

Such osteotomies can be technically challenging and difficult to perform. Further, the consequences and potential complications from such surgical procedures include delayed union, malunion, nonunion, excessive shortening of the first metatarsal, necrosis, hardware failure, infection and prolonged protected ambulation.

Percutaneous distal metatarsal osteotomy (PDMO) is a minimally invasive surgical technique for the correction of Hallux Valgus deformity. PDMO has gained popularity over other more invasive techniques such as distal chevron osteotomy. Advantages of PDMO include smaller scars, less post-operative pain, quicker recovery and reduced risk of infections and wound complications.

This technique involves conducting the osteotomy percutaneously using a burr and laterally translating the distal metatarsal head relative to the proximal metatarsal body and percutaneous insertion of one or more screws to fix the head relative to the body. Screw placement is facilitated by initially using a k-wire to provide temporary fixation of the metatarsal body and head fragments. The k-wire then guides a cannulated screw into position for cortical fixation of the head and body fragments.

Accurate placement is important because if the k-wire and the screw are placed too distally then it may crack the cortical bone. If the k-wire is too proximal then it may be oriented at an angle that is so acute it may miss the head fragment altogether.

Inserting the k-wire while maintaining the correct alignment in three axes can be difficult for a surgeon to achieve reliably. Also, the process can be time consuming and may involve extensive use of fluoroscopy. Also, the process is a two-handed operation requiring one of the surgeon's hands for wire placement and the other for maintaining the relative position of the metatarsal head fragment.

Thus, there is a need for a hallux valgus repair technique that is precise and reproducible. Ideally, there is a need for a technique that addresses these demands and that is performed by a minimally invasive approach.

Any discussion of background art throughout the specification should in no way be considered as an admission that any of the documents or other material referred to was published, known or forms part of the common general knowledge.

### SUMMARY OF THE INVENTION

In one aspect, the present invention provides a fixation device for fixing bone head and bone body fragments in an osteotomy procedure, the device including:
a first screw including a shank and an external thread for cortical fixation in the bone body fragment;
the first screw including an opening extending transversely through the shank; a second screw including a shank and an external thread for cortical fixation in the bone head fragment for relative fixation of the bone body fragment and the bone head fragment;
the shank of the second screw being locatable within the transverse opening of the first screw.

The provision of the opening extending transversely through the shank of the first screw for receiving the second screw therethrough facilitates accurate and reproducible placement of the second screw. Also, the design of the device facilitates efficient, percutaneous fixation of the distal metatarsal osteotomy for Hallux Valgus correction.

In embodiments the transverse opening comprises a longitudinal passage having a central axis between about 80 degrees and about 100 degrees and preferably about 90 degrees to a central longitudinal axis of the first screw.

In embodiments the transverse opening extends linearly through the shank from a first side to a second side.

In embodiments the external thread of the first screw is a distal external thread.

In embodiments the first screw includes proximal and distal external threads.

It should be appreciated that in embodiments in which the first screw, or otherwise referred to herein as the fixation screw, includes only a distal external thread or includes both a distal external thread and a proximal external thread, that such embodiments can be regarded as providing bi-cortical fixation in the bone body fragment.

By reference to an exemplary application in a hallux valgus correction procedure, the first screw is adapted for fixation mediolaterally in the metatarsal body fragment by insertion through the near or medial cortex and the distal thread is positively fixed into the lateral or far-cortex of the metatarsal bone body fragment. In embodiments in which the head of the screw, which has a smooth outer surface or at least does not comprise a thread, the head of the screw is located in a relatively close fit, or in a tight fit, within an opening in the medial or near-cortex of the metatarsal bone body fragment. In embodiments in which the head of the screw has a proximal external thread the proximal external thread is positively fixed within an opening in the medial or near-cortex of the metatarsal bone body fragment.

Embodiments of the first screw comprising only a distal thread and no proximal thread can be regarded as providing bi-cortical fixation in the bone body fragment. Such bi-cortical fixation including far cortical fixation by the distal thread embedded in the cortex and near cortical fixation by the location of the head of first screw in a relatively close fit, or in a tight fit, within an opening in the near-cortex. Far cortical fixation by the distal thread embedded in the cortex prevents axial movement of the first screw relative to the bone body fragment. Near cortical fixation by the location of the head of first screw, externally threaded or not, within an opening in the near-cortex prevents pivoting movement of the first screw about the point of far-cortical fixation.

In embodiments the second screw includes proximal and distal external threads for oblique cortical fixation in the bone body fragment and in the bone head fragment.

It should be appreciated that positive fixation of the proximal thread of the second screw into the near cortex enhances bi-cortical fixation of the fixation device.

In embodiments the first screw includes a second opening extending transversely through the shank of the first screw.

In embodiments the first screw includes an axial, threaded opening for receiving a threaded lug for engagement with the shank of the second screw. In a preferred embodiment the axial threaded opening is in a head of the first screw and opens into the transverse opening to permit the lug to touch the shank of the second screw to form a rigid connection therebetween.

In embodiments the second transverse opening comprises a longitudinal passage having a central axis between about 80 degrees and about 100 degrees and preferably about 90 degrees to a central longitudinal axis of the first screw.

In embodiments the second transverse opening extends linearly through the shank from a first side to a second side.

In embodiments, the device includes a third screw including a shank and an external thread for cortical fixation in the bone head fragment for relative fixation of the bone body fragment and the bone head fragment, the shank of the third screw being locatable within the second transverse opening of the first screw.

In embodiments the third screw includes proximal and distal external threads for oblique cortical fixation in the bone body fragment and in the bone head fragment.

The provision of the second opening extending transversely through the shank of the first screw for receiving the third screw therethrough facilitates accurate and reproducible placement of the third screw.

In embodiments, the central axes of the first and second transverse openings are angularly displaced from one another. Preferably the central axes of the first and second transverse openings are angularly displaced from one another from a lateral perspective preferably by 5 to 10 degrees.

In embodiments the first screw includes an intermediate external thread between the proximal and distal external threads.

In embodiments a head at the proximal end of the first screw has a proximal face that is part transversely planar at about 90 degrees to the longitudinal axis and part chamfered.

In embodiments the chamfered part is between 15 degrees and 45 degrees and preferably at about 30 degrees to the transversely planar part.

In embodiments the fixation device is for connecting bone head and bone body fragments in a distal metatarsal osteotomy procedure and wherein the first screw is adapted for cortical fixation, preferably bi-cortical fixation, in the proximal body fragment at about 30 degrees to the normal of the longitudinal axis of the proximal bone body fragment.

In embodiments the major diameter of the thread of the first screw is less than or equal to the external diameter of the shank.

In embodiments the major diameter of the distal thread of the first screw is less than or equal to the external diameter of the shank.

In embodiments the major diameter of the proximal thread of the first screw is greater than the external diameter of the shank.

In embodiments the external diameter of the shank of the second screw and the internal diameter of the transverse aperture are sized for the shank to be locatable in a tight fit within the transverse aperture.

In embodiments the major diameter of the distal thread of the second screw is less than or equal to the external diameter of the shank.

In embodiments the major diameter of the proximal thread of the second screw is greater than the external diameter of the shank.

In embodiments the distal thread of the second screw is adapted for fixation in the lateral cortex of the bone head fragment. In embodiments the distal thread of the third screw is adapted for fixation in the lateral cortex of the bone head fragment.

In embodiments the proximal thread of the second screw is adapted for fixation in the medial cortex of the bone body fragment. In embodiments the proximal thread of the third screw is adapted for fixation in the medial cortex of the bone body fragment.

In embodiments the fixation device is for connecting first metatarsal bone head and bone body fragments in a hallux valgus osteotomy procedure wherein the first screw is adapted for distal fixation in the cortex, preferably the medial and lateral cortex of the bone body fragment, and the second screw is adapted for proximal oblique fixation in the medial cortex of the bone body fragment, for distally traversing the lateral cortex of the bone body fragment and for entering the lateral cortex of the bone head fragment for relative fixation of the bone body and bone head. The proximal thread of the second screw is adapted for proximal oblique fixation in the medial cortex of the bone body fragment. The distal thread of the second screw is adapted for distal fixation in the lateral cortex of the bone head. The shank of the second screw traverses the lateral cortex of the bone body fragment.

In another aspect, the invention provides a fixation screw for use in connecting metatarsal bone head and bone body fragments in a distal metatarsal osteotomy procedure, the screw including:

a shank and an external thread for cortical fixation in the bone body fragment; a transverse opening extending transversely through the shank for receiving therewithin a second screw for oblique cortical fixation in the bone body fragment and in the bone head fragment for relative fixation of the bone body and bone head fragments.

In embodiments the screw includes a distal external thread for mediolateral far-cortical fixation in the bone body fragment.

In embodiments the screw includes proximal and distal external threads for mediolateral bi-cortical fixation in the bone body fragment.

As mentioned above, it should be appreciated that embodiments in which the fixation screw includes only a distal external thread or includes both a distal external thread and a proximal external thread, that such embodiments can be regarded as providing bi-cortical fixation in the bone body fragment. Such bi-cortical fixation including far cortical fixation by the distal thread embedded in the far cortex and near cortical fixation by the location of the head of first screw, externally threaded or not, within an opening in the near-cortex.

In embodiments the screw includes a second transverse opening extending transversely through the shank for receiving a third screw for oblique cortical fixation in the bone body fragment and in the bone head fragment for relative fixation of the bone body and bone head fragments.

In embodiments the transverse openings each comprise a longitudinal passage having a central axis between about 80 degrees and about 100 degrees and preferably about 90 degrees to a central longitudinal axis of the screw.

In embodiments the first transverse opening extends linearly through the shank from a first side to a second side.

In embodiments the second transverse openings extends linearly through the shank from a first side to a second side.

In embodiments, the central axes of the first and second transverse openings are angularly displaced from one another. Preferably the central axes of the first and second transverse openings are angularly displaced from one another from a lateral perspective preferably by 5 to 10 degrees.

In embodiments the transverse openings are defined within the shank of the screw by a smooth cylindrical surface.

In embodiments a head at the proximal end of the first screw has a proximal face that is part transversely planar at about 90 degrees to the longitudinal axis and part chamfered.

In embodiments the chamfered part is at about 30 degrees to the transversely planar part.

In embodiments the fixation screw is adapted for bi-cortical fixation at about 30 degrees to the normal of the longitudinal axis of the proximal body fragment.

In embodiments the major diameter of the distal thread of the first screw is less than or equal to the external diameter of the shank.

In embodiments the major diameter of the proximal thread of the first screw is greater than the external diameter of the shank.

In embodiments the external diameter of the shank of the second screw and the internal diameter of the transverse aperture are sized for the shank to be locatable in a tight fit within the transverse aperture.

In embodiments the fixation screw includes an axial, threaded opening for receiving a threaded lug for engagement with the shank of the second screw. In a preferred embodiment the axial threaded opening is in a head of the first screw and opens into the transverse opening to permit the lug to touch the shank of the second screw to form a rigid connection therebetween.

In another aspect, the invention provides a fixation screw assembly for use in connecting metatarsal bone head and bone body fragments in a distal metatarsal osteotomy procedure, the screw assembly including the first screw in accordance with the above aspect of the invention and its embodiments and a second screw receivable within the transverse opening extending transversely through the shank for oblique cortical fixation in the bone body fragment and in the bone head fragment for relative fixation of the bone body and bone head fragments.

In embodiments the screw assembly includes a third screw receivable within a second transverse opening extending transversely through the shank for oblique cortical fixation in the bone body fragment and in the bone head fragment for relative fixation of the bone body and bone head fragments.

In another aspect, the invention provides a system for fixation of first metatarsal bone head and bone body fragments in a hallux valgus osteotomy procedure, including:

a first screw including a shank and an external thread for cortical and mediolateral fixation in the bone body fragment;
one or more openings extending transversely through the shank of the first screw; and
a jig mountable to the first screw and including one or more guides to align with the one or more transverse openings through the first screw.

In embodiments the screw includes proximal and distal external threads for mediolateral bi-cortical fixation in the bone body fragment.

In embodiments the screw includes a second transverse opening extending transversely through the shank and the jig includes a second guide align with the second transverse opening through the first screw.

In embodiments each of the transverse openings in the first screw comprises a longitudinal passage and each of the guides includes a longitudinal guide passage, wherein the longitudinal passage of each of the transverse openings is axially aligned with a respective one of the longitudinal guide passages when the jig is mounted to the first screw.

In embodiments, the axes of the guides are angularly displaced from one another. Preferably the axes of the guides are angularly displaced from one another from a lateral perspective, and preferably by 5 to 10 degrees.

In another aspect, the invention provides an orthopaedic jig for use in connecting first metatarsal bone head and bone body fragments in a distal metatarsal osteotomy procedure, the jig including:

a jig body;
an intermediate stabilisation mount for coupling with a first screw fixated cortically and mediolaterally in the metatarsal bone body fragment
a proximal stabilisation device including a guide for receiving a threaded wire inserted into the metatarsal bone body fragment or medial cuneiform bone for proximal stabilisation of the jig body;
a distal stabilisation device including a guide for receiving a threaded wire inserted into the proximal phalanx bone for distal stabilisation of the jig body; an adjustable support mounted to the jig body for engaging the metatarsal bone head fragment intermediate of the proximal and distal stabilisation mounts and adjusting the lateral translation of the bone head fragment relative to the bone body fragment.

Embodiments of the invention facilitate efficient, accurate and reproducible percutaneous fixation of the proximal metatarsal body fragment and distal metatarsal head fragment in Hallux Valgus osteotomy surgery. The jig facilitates a step-by-step and relatively rapid approach to percutaneous hallux valgus osteotomy and also helps reduce the use of fluoroscopy. The jig allows fixation to be efficient, reliable and reproducible through the use of specifically designed instrumentation.

In embodiments the adjustable support includes a metatarsal bone head fragment interface that is spaced apart from the body of the jig wherein the distance between the interface and the jig body is adjustable.

In embodiments the interface is supported by a rod threadably coupled to the jig body whereby relative rotation between the rod and the jig body causes axial translation of the rod and the interface relative to the jig body. In an embodiment, the rod is threadably coupled to a manually rotatable member that is captured by the jig body wherein manual rotation of the rotatable member causes the axial translation of the rod and the interface relative to the jig body.

In embodiments the interface is linearly movable, preferably vertically, relative to the rod for alignment with the metatarsal bone head fragment.

In embodiments the adjustable support is movably coupled to the jig body for repositioning the adjustable support relative to the jig body between the proximal and distal ends thereof. Preferably, the adjustable support is movably coupled to a mount that is movable relative to the jig body for repositioning the adjustable support relative to the jig body.

In embodiments the adjustable support is movable longitudinally relative to the jig body.

In embodiments the jig body includes a guide adapted to guide a second screw to align with a transverse opening through a shank of the first screw fixated cortically and mediolaterally in the metatarsal bone body fragment. Preferably, wherein the second screw includes a shank and proximal and distal external threads for oblique cortical fixation in the bone body fragment and in the bone head fragment for relative fixation of the bone body and bone head fragments.

In embodiments, the jig body includes a second guide adapted to guide a third screw to align with another transverse opening through the shank of the first screw fixated bi-cortically and mediolaterally in the metatarsal bone body fragment. Preferably, wherein the third screw includes a shank and proximal and distal external threads for oblique cortical fixation in the bone body fragment and in the bone head fragment for relative fixation of the bone body and bone head fragments.

In embodiment, the jig includes a jig positioning guide member including a radio luminescent guide adapted to overlay the proximal metatarsal bone body fragment and the bone head fragment.

In another aspect, the invention provides a surgical method for fixing bone head and bone body fragments in an osteotomy procedure for correcting hallux valgus deformity, the method including:

inserting a first screw mediolaterally in a proximal metatarsal bone body fragment for cortical fixation,
inserting a second screw proximally and obliquely through the medial cortex of the proximal metatarsal bone body fragment and through an opening extending transversely through the shank of the first screw and distally traversing the lateral cortex of the bone body fragment and entering the lateral cortex of the bone head fragment for relative fixation of the bone body and bone head fragments.

In embodiments, the method includes inserting a third screw proximally and obliquely through the medial cortex of the proximal metatarsal bone body fragment and through another opening extending transversely through the shank of the first screw and distally traversing the lateral cortex of the bone body fragment and entering the lateral cortex of the bone head fragment for relative fixation of the bone body and bone head fragments.

In embodiments the method includes:

supporting a jig on the first screw fixated cortically and mediolaterally in the metatarsal bone body fragment;
stabilising a proximal end of the jig to the proximal metatarsal bone body fragment or medial cuneiform with a threaded wire inserted into the metatarsal bone body fragment or medial cuneiform bone and coupled to the jig;
stabilising a distal end of the jig to the proximal phalanx bone with a threaded wire inserted into the proximal phalanx bone and coupled to the jig;
engaging the metatarsal bone head fragment with an adjustable support mounted to the jig body intermediate the proximal and distal ends of the jig; and
adjusting the lateral translation of the bone head fragment relative to the bone body fragment with the adjustable support.

In embodiments a guide hole drilled for each of the second and third screws is guided by the jig through the proximal metatarsal bone body fragment and through the transverse openings through the first screw and into the lateral cortex of the bone head fragment.

### BRIEF DESCRIPTION OF THE FIGURES

The invention will now be described in more detail with reference to embodiments of the invention illustrated in the accompanying drawings, wherein:

FIG. 1 illustrates a dorsoplantar view of a first metatarsal including an implanted fixation device including a first screw, a second screw and a third screw for connecting the first metatarsal bone head and body fragments in an osteotomy procedure in accordance with an embodiment of the invention;

FIG. 2 illustrates a perspective view of the first screw, second screw and third screw of the fixation device of FIG. 1 with the second and third screws positioned within transverse apertures of the first screw;

FIG. 3 illustrates a mediolateral view of the implanted fixation device of FIG. 1;

FIG. 4 illustrates a mediolateral view of the first metatarsal and the line of a chevron osteotomy for the separation of the first metatarsal into the proximal body fragment and the distal head fragment for subsequent lateral translation in a procedure for hallux valgus correction;

FIG. 5 illustrates a perspective view of the first bone screw component of the fixation device of FIG. 1 including proximal and distal threads and a star-shaped recess for receiving a complementarily shaped driver therewithin;

FIG. 6 illustrates a perspective view of another embodiment of the first bone screw component further including an intermediate thread;

FIG. 7 illustrates a front view of the first bone screw embodiment of FIG. 5;

FIG. 8 illustrates a front view of the first bone screw embodiment of FIG. 6;

FIG. 9 illustrates a frontal cross section view of the first bone screw embodiment of FIG. 5;

FIG. 10 illustrates a frontal cross section view of the first bone screw embodiment of FIG. 6;

FIG. 11 illustrates a top view of the first bone screw embodiment of FIG. 5;

FIG. 12 illustrates a top view of the first bone screw embodiment of FIG. 6;

FIG. 13 illustrates a perspective view of another embodiment of the first bone screw component including only a distal thread and first and second transverse openings for receiving second and third bone screws therewithin and a slot shaped recess for receiving a complementarily shaped driver therewithin;

FIG. 14 illustrates a frontal cross section view of the first bone screw of FIG. 13;

FIG. 15 illustrates a front view of the first bone screw of FIG. 13;

FIG. 16 illustrates another perspective view of the first bone screw of FIG. 13;

FIG. 17 illustrates a front view of another embodiment of the first bone screw component including only a distal thread and one transverse opening for receiving a second bone screw therewithin;

FIG. 18 illustrates a frontal cross section view of the first bone screw of FIG. 17;

FIG. 19 illustrates perspective view of the first bone screw of FIG. 17;

FIG. 20 illustrates a frontal cross-section view of a second bone screw component of the fixation device of FIG. 1 including a shank and proximal and distal external threads;

FIG. 21 illustrates front view of the second bone screw of FIG. 20;

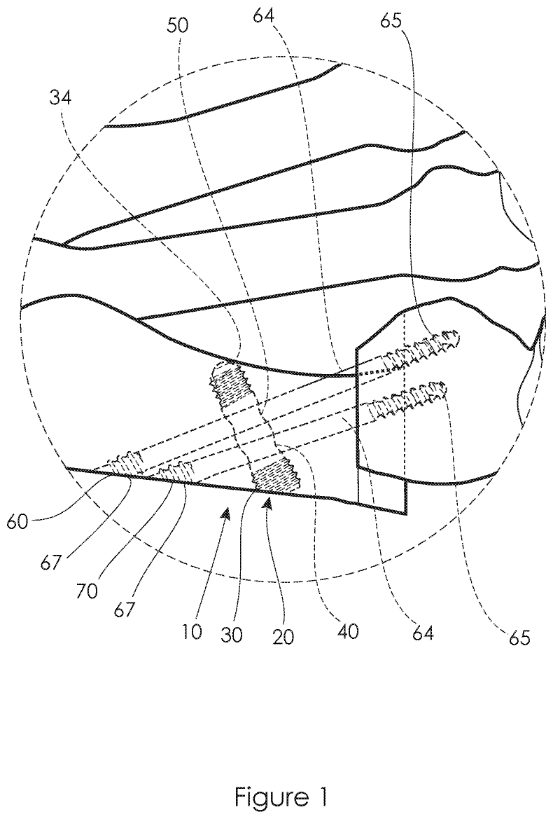

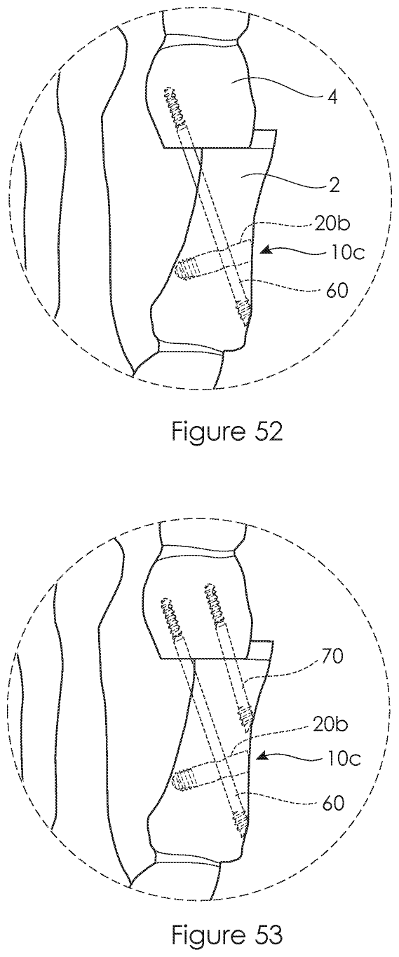

FIG. 20 illustrates a frontal cross-section view of a second bone screw component of the fixation device of FIG. 1 including a shank and proximal and distal external threads;

FIG. 21 illustrates front view of the second bone screw of FIG. 20;

FIG. 22 illustrates a top view of the second bone screw of FIG. 20;

FIG. 23 illustrates a frontal cross section view of the third bone screw component of the fixation device of FIG. 1 including a shank and proximal and distal external threads;

FIG. 24 illustrates front view of the second bone screw of FIG. 23;

FIG. 25 illustrates a top view of the second bone screw of FIG. 23;

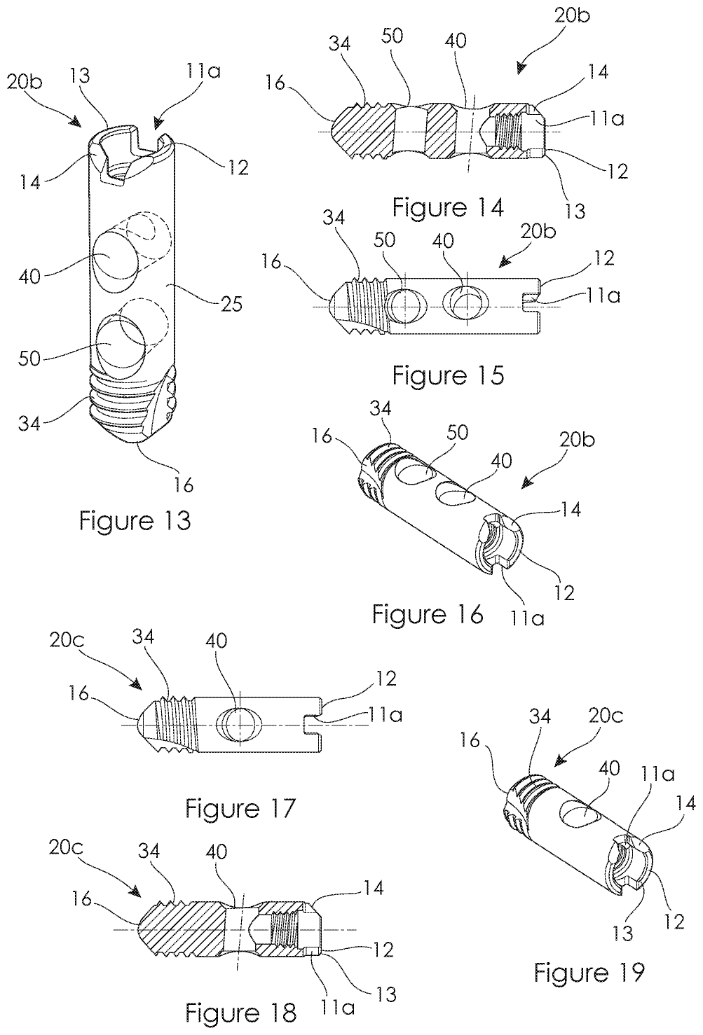

FIG. 26 illustrates a perspective view of the first bone screw, second bone screw and third bone screw components of a fixation device comprising the first bone screw embodiment of FIGS. 13 to 16 and with the second and third screws positioned within the transverse apertures of the first screw;

FIG. 27 illustrates a perspective view of the first metatarsal including the first bone screw fixed bi-cortically in the proximal metatarsal body fragment and an orthopaedic jig in accordance with an embodiment of the invention coupled thereto;

FIG. 28 illustrates a dorsoplantar view of the first bone screw and the orthopaedic jig of FIG. 10;

FIG. 29 illustrates a perspective view of the jig coupled to the first bone screw and including a threaded guidewire located within a guide hole of a proximal stabilisation device;

FIG. 30 illustrates a perspective view of the proximal stabilisation support of the jig including the threaded guidewire located within the guide hole and a locking screw including a manually rotatable head of a threaded member adapted for locking the guidewire within the guide hole;

FIG. 31 illustrates a perspective view of a distal portion of the jig including an adjustable support adapted for engaging the metatarsal head fragment and an intermediate stabilisation mount adapted to be coupled to the first bone screw and including a guide hole for a threaded guidewire a distal stabilisation device and a locking screw including a rotatable knurled head coupled to a threaded rod adapted for locking the guidewire within the guide hole;

FIG. 32 illustrates a perspective view of a component of the adjustable support including a metatarsal head interface supported by a rod for threaded coupling with the jig;

FIG. 33 illustrates a reverse perspective view of the metatarsal head interface and rod of FIG. 32;

FIG. 34 illustrates a perspective view of the jig of FIG. 27 coupled to the first bone screw and to a proximal guide wire and a distal guidewire and the adjustable support coupled to the jig with the metatarsal head interface thereof in engagement with the metatarsal head;

FIG. 35 illustrates a perspective view of the jig of FIG. 27 proximally stabilised to the proximal metatarsal bone fragment and distally stabilised to the proximal phalanx as illustrated in FIG. 34 wherein a proximal drill bit guide is adapted to guide insertion of a drill bit at an oblique angle into medial cortex of the proximal metatarsal body fragment and through one of the openings extending transversely through the shank of the first bone screw;

FIG. 36 illustrates a perspective view of the jig of FIG. 27 wherein after the drill bit forms a guide hole as illustrated in FIG. 35, the second bone screw is driven therein for fixation in the medial cortex of the proximal metatarsal body and for either distal fixation, or to at least pass through the distal lateral cortex of the proximal metatarsal fragment at a distal end thereof and for fixation in the metatarsal head fragment;

FIG. 37 illustrates a dorsoplantar view of the metatarsal and the first screw and the second screw;

FIG. 38 illustrates a dorsoplantar view of the metatarsal and first screw and the second and third screws;

FIG. 39 illustrates a side view of a 2.5 mm drill bit including a smaller diameter 1.6 mm section about 7 mm in length from the tip.

FIG. 40 illustrates an alternative embodiment of the metatarsal head fragment interface of the adjustable support.

FIG. 41 illustrates a perspective view of an alternative embodiment of the metatarsal head fragment interface of the adjustable support, wherein the metatarsal head fragment is received within a recess of the interface which is slidably coupled to the supporting rod;

FIG. 42 illustrates a perspective view of the interface and supporting rod of FIG. 29 wherein the supporting rod is coupled to the jig in accordance with another embodiment thereof;

FIG. 43 illustrates a perspective view of the first metatarsal including the first bone screw fixed bi-cortically in the proximal metatarsal body fragment and an orthopaedic jig in accordance with another embodiment of the invention coupled thereto, the jig being stabilised proximally and distally relative to the proximal metatarsal and the proximal phalanx by threaded guidewires and by the head fragment interface of FIG. 41;

FIG. 44 illustrates a perspective view of the jig of FIG. 43 wherein a driver is employed to drive a second screw into the proximal metatarsal bone body fragments;

FIG. 45 illustrates a dorsoplantar view of the metatarsal and first screw and one second screw and the metatarsal head fragment interface of FIG. 41.

FIG. 46 illustrates a perspective view of the first metatarsal including the first bone screw fixed bi-cortically in the proximal metatarsal body fragment and an orthopaedic jig in accordance with another embodiment of the invention coupled thereto, wherein the jig is comprised of a proximal member and a distal member and a jig positioning guide member;

FIG. 47 illustrates a top view of the jig of FIG. 46 including a jig positioning guide member including radio luminescent guides adapted to overlay the proximal metatarsal body fragment and the bone head fragment;

FIG. 48 illustrates a perspective view of the first metatarsal including the first bone screw fixed bi-cortically in the proximal metatarsal body fragment and an orthopaedic jig in accordance with another embodiment of the invention coupled thereto, wherein the jig is comprised of a proximal member and a distal member and wherein the distal member of the jig is represented in broken lines;

FIG. 49 illustrates a top view of the jig of FIG. 46 including a jig positioning guide member including radio luminescent guides adapted to overlay the proximal metatarsal body fragment and the bone head fragment;

FIG. 50 illustrates a rear perspective view of a portion of the jig of FIG. 46;

FIG. 51 illustrates a perspective view of a portion of the jig of FIG. 46 where the distal member of the jig is represented in solid lines;

FIG. 52 illustrates a dorsoplantar view of a first metatarsal including an implanted fixation device including the first screw of the embodiment of FIG. 17 wherein a second bone screw according to the embodiment of FIG. 20 is received through the transverse opening and distally traverses the lateral cortex of the bone body fragment for oblique cortical fixation in the bone body fragment and in the bone head fragment;

FIG. 53 illustrates the device of FIG. 52 and further including a third screw according to the embodiment of FIG. 23 for oblique cortical fixation in the bone body fragment and in the bone head fragment for relative fixation of the bone body and bone head fragments.

### DETAILED DESCRIPTION

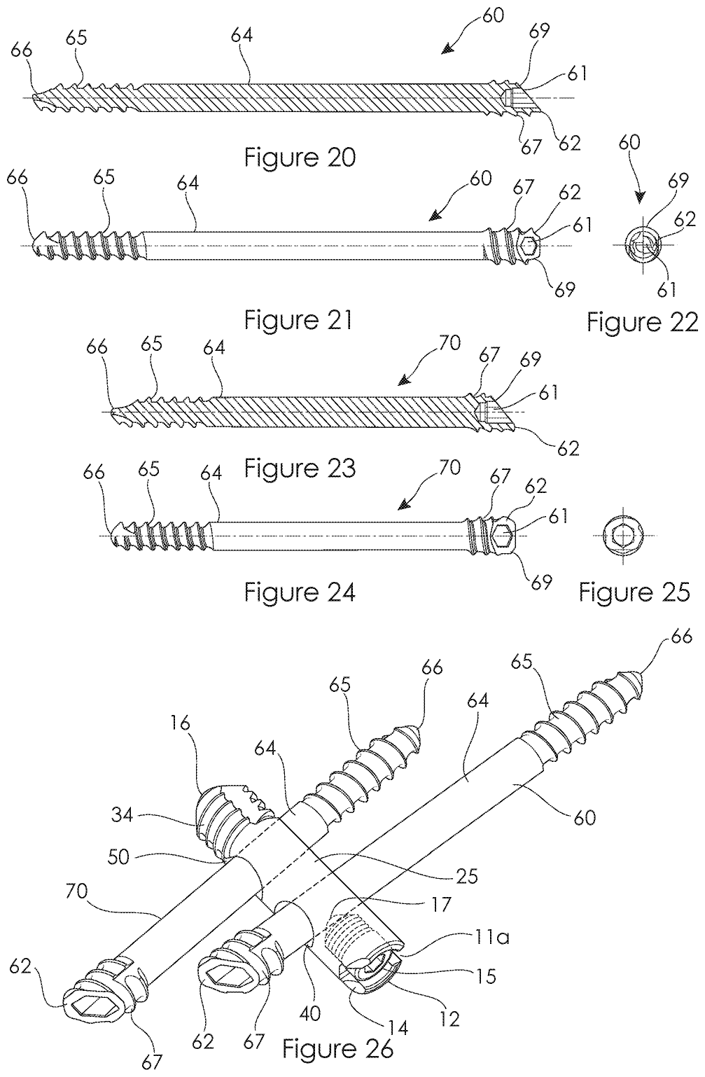

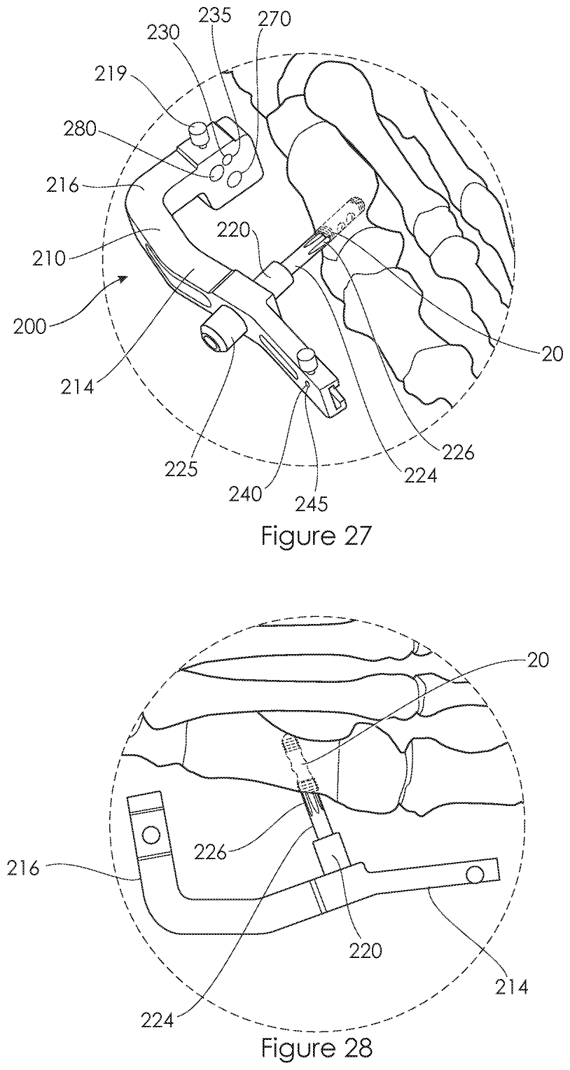

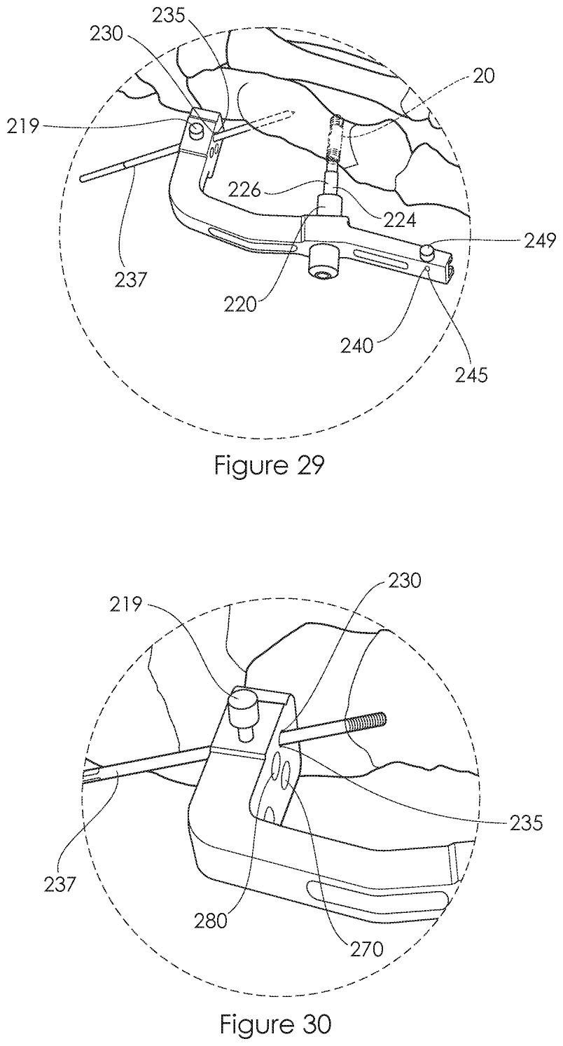

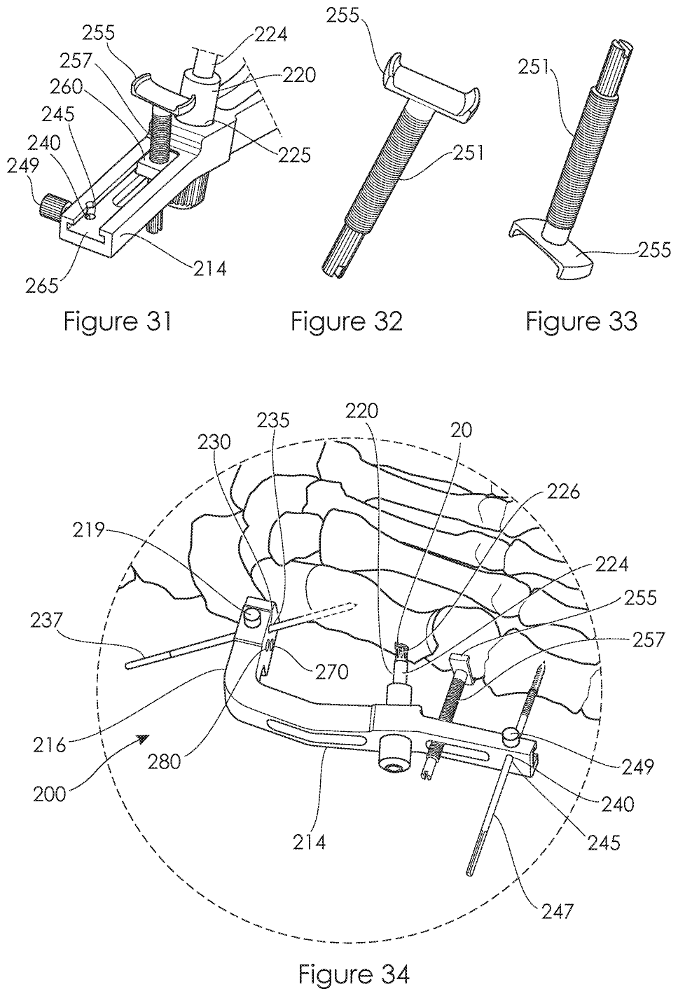

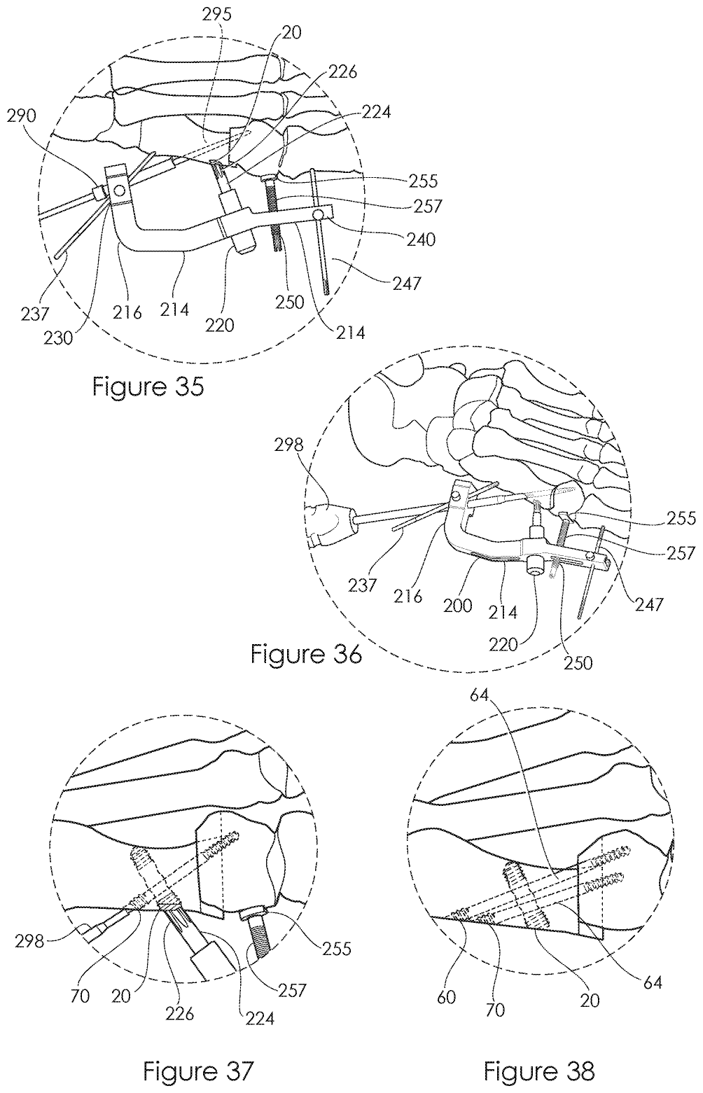

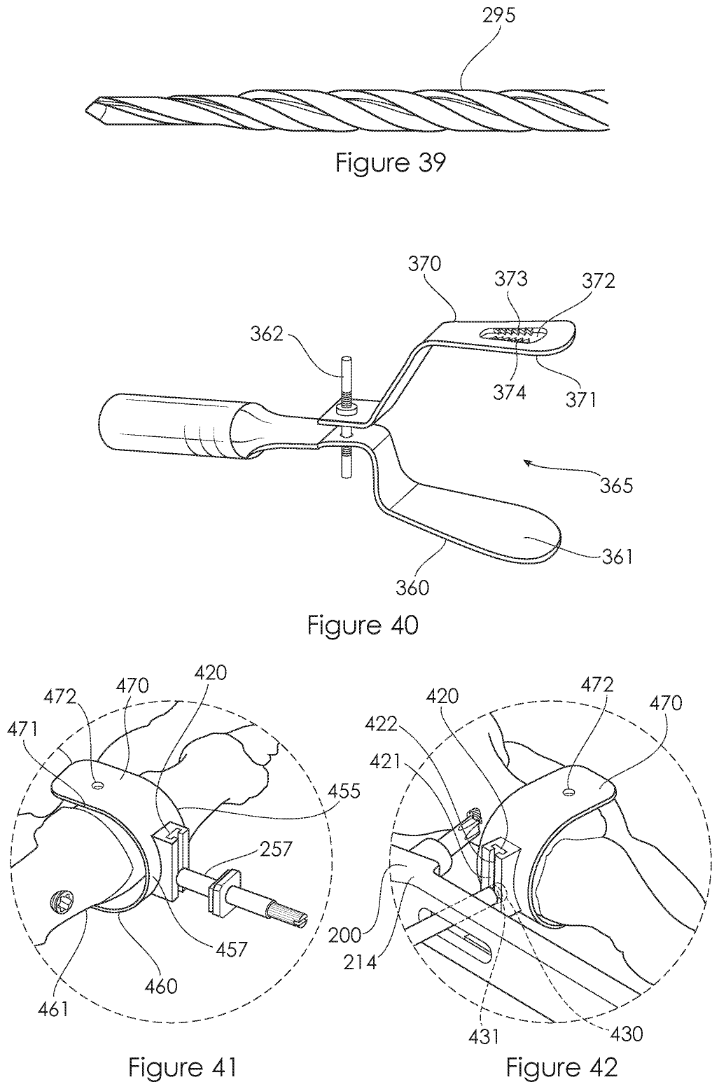

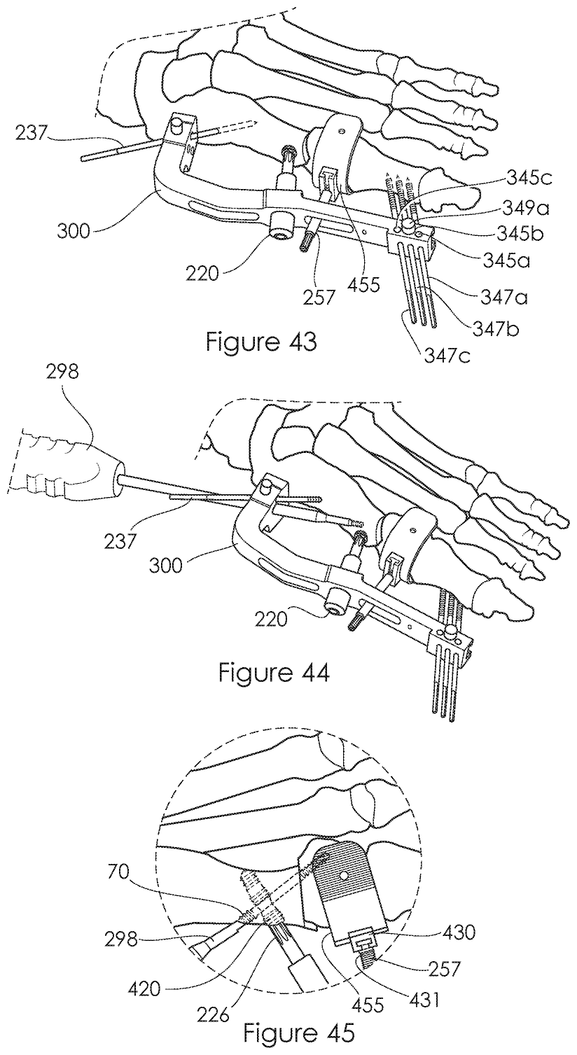

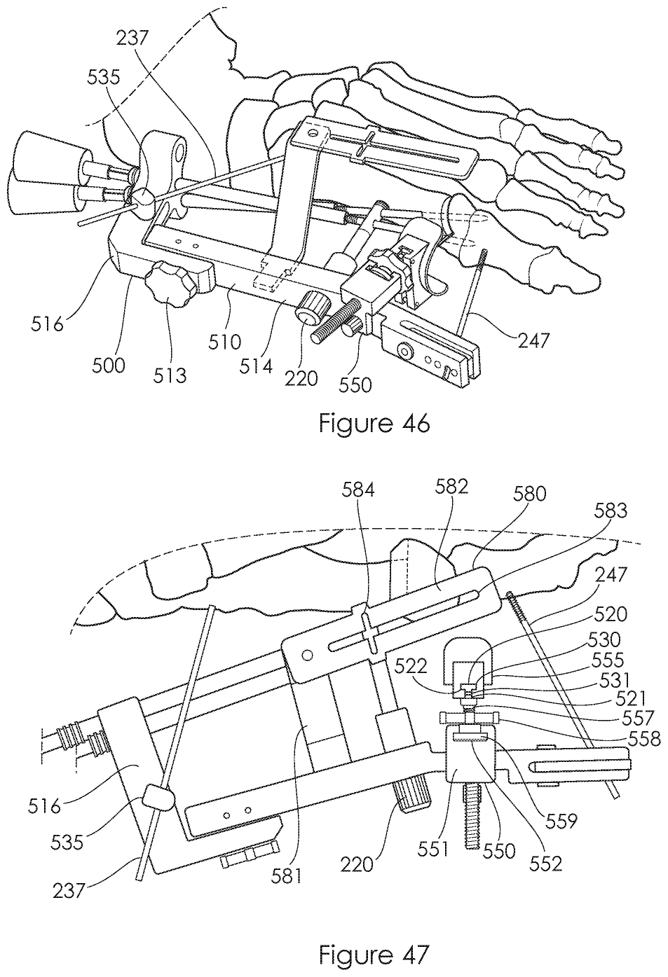

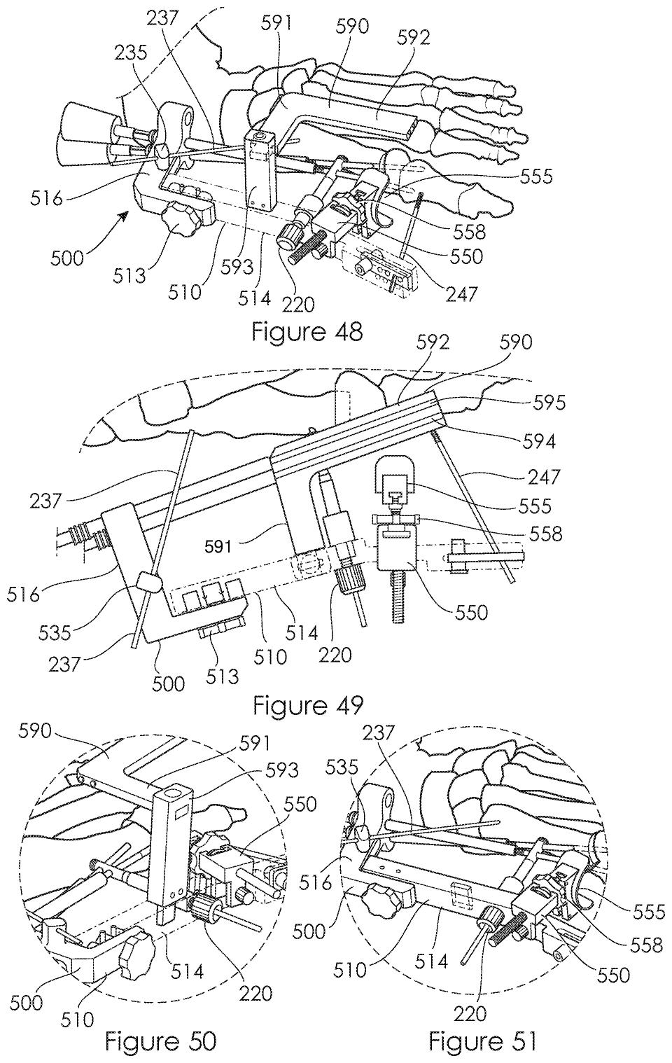

FIGS. 1 to 37 illustrate embodiments of the invention including a fixation device 10, an orthopaedic jig 200 and a surgical method for connecting bone head and body fragments in an osteotomy procedure. The invention is particularly suitable for use in relation to percutaneous distal metatarsal osteotomy (PDMO) for the correction of Hallux Valgus deformity. FIG. 4 illustrates a mediolateral view of the first metatarsal 1 and proximal phalanx 6 and the line of a chevron osteotomy 3 for the separation of the first metatarsal 1 into a proximal body fragment 2 and a distal head fragment 4 for subsequent lateral translation in a procedure for hallux valgus correction. Accordingly, in the Figures, the fixation device 10, orthopaedic jig 200 and the surgical method are illustrated in the context of their use in the fixation of a proximal metatarsal bone body fragment with a realigned distal metatarsal bone head fragment in an osteotomy procedure.

Referring to FIGS. 1 to 5, the device 10 includes a first screw 20 comprising a shank 25 extending between a head 12 and a tip 16. The head 12 includes an axially facing planar surface 13 that is substantially perpendicular to the longitudinal axis of the shank 25. Also, for reasons that will become apparent, the head 12 includes a chamfered part 14 that is at about 30 degrees to the axially facing planar surface 13. The chamfered part 14 and the axially facing planar surface 13 meet at about a midline through the head 12 of the first screw 20. The head 12 also includes a star-drive shaped recess 11 adapted to receive a complementary shaped tip of a driver. It is to be appreciated that others shaped recesses such as a hex-drive recess and driver may be adopted.

The shank 25 includes a proximal external thread 30 and a distal external thread 34. The shank 25 is cylindrical with a substantially uniform diameter of about 4.5 mm and is formed of a titanium alloy. The overall length of the first screw 20 from the head 12 to the tip 16 varies between about 12 mm and 26 mm. The distal external thread 34 has a major diameter that is equal to or less than the external diameter of the shank 25. The proximal thread 30 has a major diameter that is greater than the diameter of the shank 25. The first screw 20 is adapted for bi-cortical fixation in the bone body fragment, which in the figures is a proximal metatarsal bone body fragment.

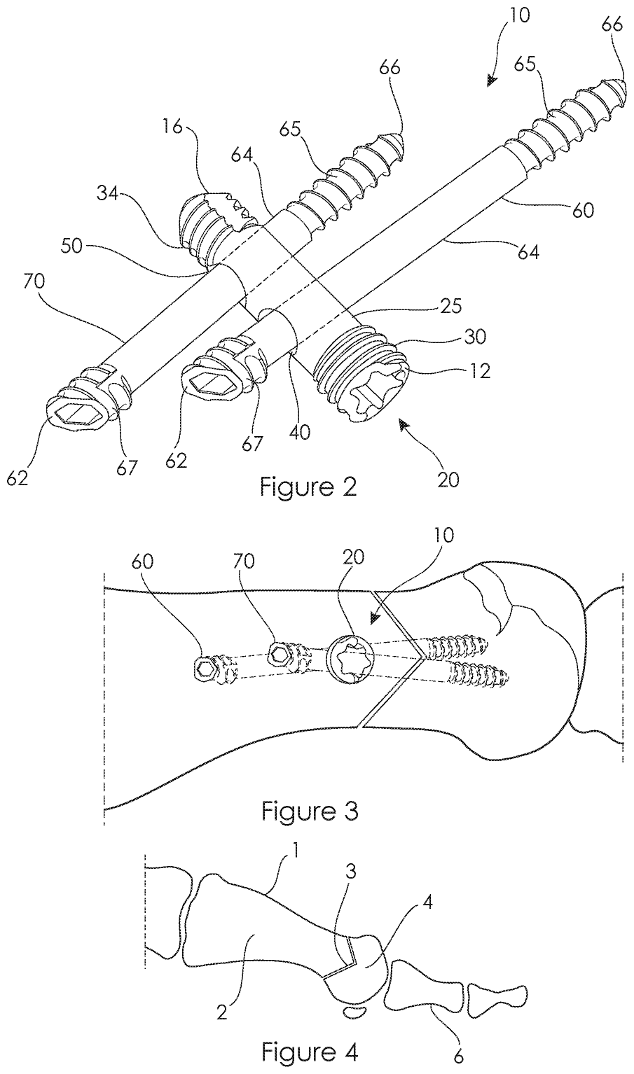

Referring to FIGS. 2 to 5, the first screw 20 includes at least one and preferably two openings 40, 50 extending transversely through the shank 25. The transverse openings 40, 50 each comprise a longitudinal passage 42, 52 having a central axis between about 80 degrees and about 100 degrees and preferably about 90 degrees to a central longitudinal axis of the first screw 20. As illustrated in FIG. 2, the central axes of the transverse openings 40, 50 are radially offset relative to each other by about 5 to 10 degrees. One of the transverse openings 40 is located closer to the proximal head 12 with the longitudinal axis spaced apart from the planar face 13 by about 6.2 mm. The other one of the transverse openings 50 is located further from the proximal head 12 with the longitudinal axis spaced apart from the planar face 13 by about 11.0 mm.

FIGS. 2a, 3a, 4a and 5a, illustrate another embodiment of the first screw 120 that is largely identical to the embodiment of FIGS. 2, 3, 4 and 5 except for an intermediate thread 125 located between the proximal external thread 30 and the distal external thread 34. The intermediate thread 125 is located in the vicinity of the two transverse openings 40, 50. As can be seen in FIGS. 2a, 3a, 4a and 5a the intermediate thread 125 is between and extends over at least part of length of the 25 comprising the two transverse openings 40, 50.

Referring to FIGS. 1 and 6 to 8, the device 10 further includes at least a second screw 60 and preferably a third screw 70 that are adapted to cooperate with the first screw 10 in a manner described below. The second screw 60 and third screw 70 are preferably identical and if they differ it may only be in length. Accordingly, the identical features of the second screw 60 and the third screw 70 will be described below with reference to the second screw 60 only and in the Figures like features will be identified with like reference numerals.

Referring to FIGS. 6 to 8, the second screw 60 includes a shank 64 extending between a head 62 and a tip 66. The head 62 is bevelled such that the entire end face 69 of the head 62 is oriented at about 40 degrees to the perpendicular to the longitudinal axis of the shank 64. The head 62 also includes a hex-drive shaped recess 61 adapted to receive a corresponding shaped tip of a driver. It is to be appreciated that any other configuration such as a star-drive or square-drive recess and driver may be adopted.

The second screw 60 may be a cannulated wire guided screw or as illustrated in the Figures and in FIGS. 6 and 7 a solid and non-cannulated and is adapted for wire guided insertion into the bone body and head fragments as described in more detail below. The shank 64 includes a proximal external thread 67 and a distal external thread 65. The shank 64 is cylindrical with a substantially uniform diameter of about 2.5 mm and is formed of a titanium alloy. The overall length of the second screw 60 from the head 62 to the tip 66 varies between about 16 mm and 60 mm. The distal external thread 65 has a major diameter that is equal to or less than the external diameter of the shank 64. The proximal thread 67 has a major diameter that is greater than the diameter of the shank 25.

As illustrated in FIG. 1, the second screw 60 is adapted for oblique cortical fixation in the bone body fragment and in the bone head fragment for relative fixation of the body and head. Also, the shank 64 of each of the second screw 60 and third screw 70 are locatable within a respective one of the transverse openings 40, 50 of the first screw 20. The external diameters of the shank 64 of the second screw 60 and the third screw 70 and the internal diameters of the longitudinal passages 42, 52 of the transverse openings 40, 50 are sized for the shanks 64 to be locatable in a close-fit within the transverse openings 40, 50.

An axial, threaded opening in the head 12 is adapted to receive a threaded lug. An embodiment of the lug 15 is illustrated in FIG. 26 which shows that when the lug is screwed into the head of the first screw a distal tip 17 of the lug engages the shank 64 of the second screw 60 to form a rigid connection therebetween.

As illustrated in FIG. 22, because the central axes of the transverse openings 40, 50 are radially offset relative to each other by about 10 degrees, this means that the orientation of the second screw 60 and the third screw 70 will be slightly different and will each have a different angular displacement relative to the transverse plane. As a result, the longer second screw 60 that is inserted proximally into the metatarsal body fragment has a relatively lower proximal head 62 in the dorsoplantar direction relative to the proximal head 62 of the short third screw 70 that is inserted distally into the metatarsal body fragment. Conversely, the distal tip 66 of the longer first screw 60 is relatively higher in the dorsoplantar direction relative to the distal tip 66 of the shorter third screw 70. In other words, the proximal ends and distal ends of the second screw 60 and the third screw 70 diverge relative to an anteroposterior axis.

The first screw 20 is adapted for bi-cortical fixation in the bone body fragment, which in the figures is a proximal metatarsal bone body fragment, at about 30 degrees to the normal of the central longitudinal axis of the proximal bone body fragment. The chamfered part 14 of the head 12 is substantially flush with the external medial surface of the proximal metatarsal bone body fragment. The second screw 60 and third screw 70 are inserted proximally and laterally and at an oblique angle into the proximal metatarsal bone body fragment. When fully inserted, the proximal thread 67 is fixed in the medial cortex of the bone body fragment and the shank 64 and/or the distal thread 65 distally traverse the lateral cortex of the bone body fragment. The bevelled end face 69 of the head 62 is substantially flush with the external medial surface of the proximal metatarsal bone body fragment. The distal thread 65 of at least one or both of the second screw 60 and the third screw 70 is fixed in the lateral cortex of the bone head fragment for relative fixation of the body and head.

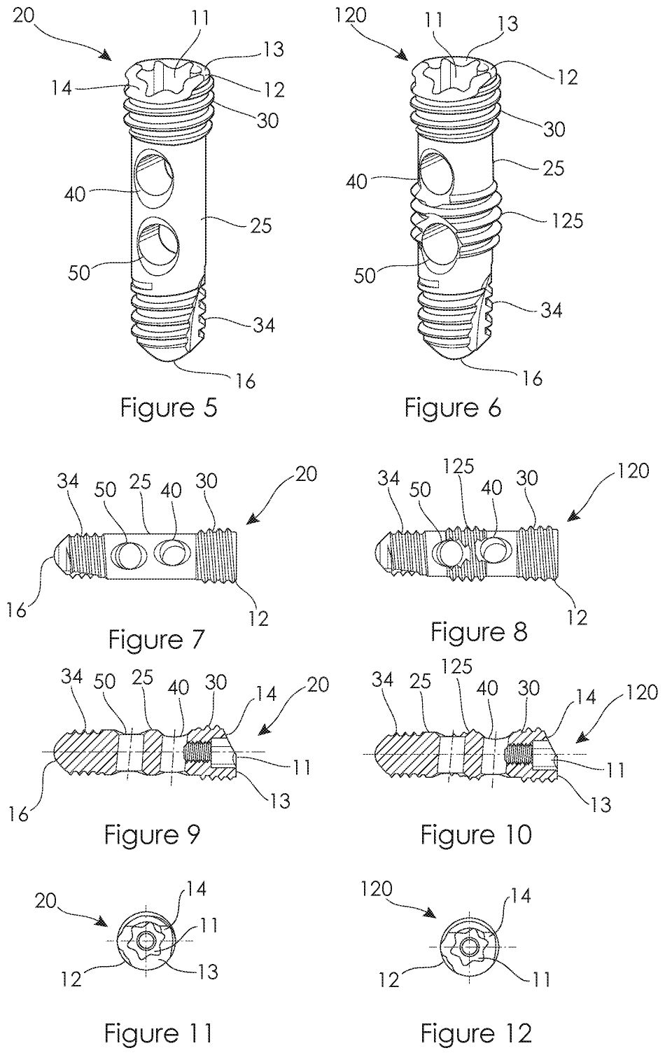

Referring to FIGS. 13 to 16, another embodiment of the first screw 20b is illustrated that in many respects is the same as the embodiment of FIG. 5 such that like features are identified with like reference numerals. The first screw 20b includes a shank 25 extending between a head 12 and a tip 16 and a pair of the transverse openings 40, 50. The head 12 includes an axially facing planar surface 13 that is substantially perpendicular to the longitudinal axis of the shank 25 and a chamfered part 14 that is at about 30 degrees to the axially facing planar surface 13. The head 12 includes a slot or a U-shaped recess 11a adapted to receive a complementary shaped tip of a driver (not shown).

FIG. 26 illustrates an embodiment of the assembled device 10b comprising the first screw 20b which also differs from other embodiments in having only the distal external thread 34. The second screw 60 and the third screw 70 are the locatable within the transverse openings 40, 50 of the first screw 20b. An axial, threaded opening in the head 12 is adapted to receive a threaded lug 15 that includes a hex shaped recess for receiving a hex-shaped drive member to screw the lug 15 into the axial, threaded opening in the head 12 of the first screw 10b. When the lug 15 is screwed into the head of the first screw a distal tip 17 of the lug engages the shank of the second screw 60 to form a rigid connection therebetween.

Unlike the embodiment of FIG. 5, the shank 25 includes only a distal external thread 34 and the proximal part of the shank has a smooth outer surface. The shank 25 is cylindrical with a substantially uniform diameter of about 4.5 mm and is formed of a titanium alloy. The overall length of the first screw 20 from the head 12 to the tip 16 varies between about 12 mm and 26 mm. The distal external thread 34 has a major diameter that is equal to or less than the external diameter of the shank 25.

Accordingly, unlike the embodiment of FIG. 5, the first screw 20b of FIGS. 13 to 16 is adapted for cortical fixation only in the lateral cortex of the proximal metatarsal bone body fragment. Yet even without a proximal external thread for the first screw 20b it has been found that there can be adequate bi-cortical fixation of the overall fixation device 10 comprising the first screw 20b and the second screw 60 and optionally a third screw 70 in the proximal metatarsal bone body fragment. When the device 10b is fixed in the proximal metatarsal bone body fragment the distal external thread 34 of the first screw 20b is fixed in the lateral cortex of the bone body fragment and the proximal external thread 67 of the second screw 60 is fixed in the medial cortex of the bone body fragment thereby providing bi-cortical fixation of the overall device 10.

Referring to FIGS. 17 to 19, another embodiment of the first screw 20c is illustrated that in many respects is the same as the first screw 20b embodiment of FIGS. 13 to 16 except that it includes only one transverse opening 40. FIGS. 52 and 53 illustrate an embodiment of the assembled device 10c comprising the first screw 20b embodiment having only the distal external thread 34.

FIG. 52 illustrates a dorsoplantar view of a first metatarsal including the fixation device including the first screw 20c of FIGS. 17 to 19 wherein the second bone screw 60 is received through the transverse opening 40 and distally traverses the lateral cortex of the bone body fragment for oblique cortical fixation in the bone body fragment and in the bone head fragment. FIG. 53 illustrates the device of FIG. 52 and further including the third screw 70 provided for oblique cortical fixation in the bone body fragment and in the bone head fragment.

Referring to FIGS. 27 to 37, an orthopaedic jig 200 in accordance with an embodiment of the invention is illustrated. The illustrated jig 200 is for use in connecting first metatarsal head and body fragments in a distal metatarsal osteotomy procedure. The jig 200 includes a body 210 preferably formed of a surgical steel or a composite such as carbon fibre or some other suitable, radiolucent material. The body 210 includes an elongated main body portion 214 and a minor body portion 216 extending transversely from a proximal end of the main body portion 214.

The elongated main body portion 214 includes an intermediate stabilisation mount 220 for coupling with a first screw, such as the first screw 20 described above, which has been fixated cortically, or bi-cortically, and mediolaterally in the metatarsal body fragment. The intermediate stabilisation mount 220 includes a rod 224 that is coupled at a proximal end 225 to the main body portion 214 and extends transversely therefrom. The other end of the rod 224 is comprised of a star-drive shaped head 226 adapted to fit the star shaped recess 11 of the first screw 20.

The transverse minor body portion 216 includes a proximal stabilisation device 230 including a guide aperture 235. The guide aperture 235 is adapted for receiving a threaded wire 237 which is inserted at an oblique angle from a proximal-medial direction into the medial cortex of the metatarsal body fragment or the medial cortex of the medial cuneiform. As illustrated in FIG. 30, a threaded locking screw 219 is adapted to lock the threaded wire 237 for proximal stabilisation of the jig body 210 relative to the proximal metatarsal bone body fragment.

As illustrated in FIGS. 27, 29 and 34, the elongated main body portion 214 extends from the proximal end to a distal end at which there is located a distal stabilisation device 240 including a guide aperture 245. As illustrated in FIG. 17, the guide aperture 245 is adapted for receiving a threaded guide wire 247 which is inserted at an oblique angle from a disto-medial direction into the medial cortex of the proximal phalanx or great toe. A threaded locking screw 249 is adapted to lock the threaded wire 247 for distal stabilisation of the jig body 210 relative to the phalanx.

As illustrated in FIGS. 31 to 37, the jig 200 further includes an adjustable support 250 mounted to the jig body 210 and, in particular, mounted to a distal end of the elongated main body portion 214 between the intermediate stabilisation mount 220 and the distal stabilisation device 240. The adjustable support 250 is adapted for engaging the metatarsal head fragment and adjusting the lateral translation/reduction of the head fragment relative to the body fragment.

The adjustable support 250 includes a metatarsal head fragment interface 255 that is spaced apart from the body 210 of the jig 200. The adjustable support 250 is mounted to the jig 200 so that the distance between the interface 255 and the jig body 210 is adjustable. As a result, the distance of the metatarsal head fragment from the jig body 210, and in particular the elongated main body portion 214 of the jig body 210, and thereby the mediolateral translation of the metatarsal head fragment relative to the proximal metatarsal bone body fragment can be adjusted with fine control. Furthermore, as the phalanx is stabilised by the threaded guidewire 247 at the distal end of the elongated main body portion 214 the phalanx and the metatarsal head fragment tend be biased towards their initial position and against the interface 255. When the adjustable support 250 is adjusted to cause the mediolateral translation of the metatarsal head fragment this movement is resisted by deflection of the distal guidewire 247. These features ensure controlled movement of the bone head fragment relative to the bone body fragment between completion of the osteotomy and fixation.

The metatarsal head fragment interface 255 of the adjustable support 250 is supported by a rod 257 that is threadably coupled to the elongated main body portion 214. Rotation of the rod 257 causes axial translation of the rod 257 and the interface 255 relative to the main body portion 214 of the jig body 210. Further adjustability of the adjustable support 250 is derived from the threaded rod 257 being coupled to a mount 260 that is movable relative to the elongated main body portion 214 of the jig body 210 for repositioning the adjustable support 250 at a desired location between the proximal and distal ends thereof and between the intermediate stabilisation mount 220 and the distal stabilisation device 240. Accordingly, the adjustable support 250 can be repositioned along the length of the jig body 210 to align with the metatarsal head fragment. In the embodiment illustrated in FIG. 31, the mount 260 is freely movable by sliding longitudinally in a recess 265 within the elongated main body portion 214. In another embodiment, the mount 260 could be selectively lockable in position relative to the elongated body portion 214.

FIG. 40 illustrates an alternative embodiment of the metatarsal head fragment interface 355 of the adjustable support 250. Like the aforementioned embodiment, the interface 355 is supported by the rod 257 that is threadably coupled to the elongated main body portion 214 of the jig 200. However, unlike the previous embodiment, the interface 355 of FIG. 40 is comprised of a pair of jaws 360, 370 adapted to be opened and closed and defining a recess 365 for receiving the metatarsal head fragment therebetween. The jaws 360, 370 each include a metatarsal contacting surface 361, 371 and are coupled by a pivot connection or, as illustrated in FIG. 40, by a clamp mechanism including a threaded rod 362 adapted to move the jaws 360, 370 and their associated contacting surfaces 361, 371 towards or apart from each other. The contacting surface 371 of one of the jaws 370 is adapted to contact the dorsal side of the metatarsal head fragment while the contacting surface 361 of the other one of the jaws 360 is adapted to contact the plantar side. The threaded rod 362 is operable to adjust the relative positions of the jaws 360, 370 to engage the metatarsal head fragment dorsally and plantarly and grip the metatarsal head therebetween.

The dorsal jaw 370 includes a longitudinal slot 372 that extends along at least a portion of the length of the metatarsal head contacting surface 371 of the dorsal jaw 370. When the jaws 360, 370 are in use to grip the metatarsal head the longitudinal slot 372 extends in a mediolateral direction. Rows of teeth 373, 374 extend along opposite sides of the slot 372 and are adapted to receive a guide wire (not shown) therebetween. In use, the guidewire is inserted into the dorsal cortex of the metatarsal head fragment and extends substantially vertically through the slot 372 between the rows of teeth 373, 374 that engage the guidewire.

The guidewire can be angularly displaced by manually translating the guidewire transversely to its longitudinal axis and back and forth within the longitudinal slot 372 between the rows of teeth 373, 374. By manually angularly displacing the guidewire the metatarsal head fragment can be rolled about a longitudinal anteroposterior axis (i.e. supinated) so that in addition to adjusting mediolateral translation the head fragment can be supinated relative to the body fragment. The rows of teeth 373, 374 are formed in opposing pairs separated by opposing gaps for receiving and maintaining the guidewire in a selected angular position within the longitudinal slot 372.

FIGS. 41 to 45 illustrate another embodiment of the metatarsal head fragment interface 455 of the adjustable support 250. Like the aforementioned embodiment, the interface 455 is supported by the rod 257 that is threadably coupled to the elongated main body portion 214 of the jig 200. However, unlike the previous embodiment, the interface 455 of FIGS. 41 to 45 is comprised of a substantially U-shaped member or saddle 456 comprised of a pair of legs 460, 470 connected by a web 457 adapted to be opened and closed and defining a recess 465 for receiving the metatarsal head fragment therebetween. The legs 460, 470 each include a metatarsal contacting surface 461, 471. Unlike in the embodiment of FIG. 40, in the embodiment of the interface 455 of FIGS. 41 to 45, the legs 460, 470 are maintained in a fixed relative position by the web 457. The contacting surface 471 of one of the legs 470 is adapted to contact the dorsal side of the metatarsal head fragment while the contacting surface 461 of the other one of the legs 460 is adapted to contact the plantar side.

The plantar side leg 460 and the dorsal side leg 470 each include an aperture 462, 472 extending between opposite sides thereof. When the metatarsal head Is within the recess 465 between the legs 460, 470 a guide wire (not shown) is located within the aperture 472 and is inserted through either or both apertures 462, 472 into the dorsal cortex of the metatarsal head fragment. The guide wire extends substantially vertically through either or both of the apertures 462, 472 to fix the metatarsal head fragment to the head fragment interface 455. In another embodiment (not shown) the interface 455 could include the longitudinal slot 372 of the embodiment of FIG. 40 within the dorsal leg 470. Like the embodiment of FIG. 40, in the embodiment of FIGS. 41 to 45 including the longitudinal slot (not shown) the guidewire can be angularly displaced by manually translating the guidewire transversely to its longitudinal axis and back and forth within the longitudinal slot between the rows of teeth thereby rolling the metatarsal head fragment about a longitudinal anteroposterior axis.

As best shown in FIGS. 41 and 42, the interface 455 includes a linear slot 420 located on the outer surface of the web 457 for receiving a complementary shaped head 430 of the rod 257. The head 430 of the rod 257 includes an annular slot 431 spaced a small distance apart from the end of the rod 257 adapted for receiving elongated and opposed flanges 421, 422 arranged either side of the slot 420. The flanges 421, 422 are slidable within the annular slot 431 to thereby allow linear relative movement of the interface 455 and the rod 257. The interface 455 is movably coupled to the rod 257 in a manner that allows the interface 455 to move vertically relative to the rod 257 and jig 200 for positioning the recess 465 to receive the metatarsal head fragment.

FIGS. 43 to 45 illustrate an embodiment of the jig 300 that in most respects is identical to the jig 200 of FIGS. 27 to 37, except that the distal end includes a distal stabilisation device 340 including a plurality of guide apertures 345a, 345b, 345c. As illustrated in FIGS. 43 to 45, the guide apertures 345a, 345b, 345c are each adapted for receiving a respective threaded guide wire 347a, 347b, 347c which are inserted at an oblique angle in a distal-medial to proximal-lateral direction into the medial cortex of the proximal phalanx or great toe. Threaded locking screws 349a, only one of which is illustrated in FIG. 43, are adapted to lock the threaded wires 347a, 347b, 347c for distal stabilisation of the jig body 310 relative to the proximal phalanx.

Having set the position of the metatarsal head fragment relative to the proximal metatarsal bone fragment, as illustrated in FIGS. 35 to 38 and 44, 45, the jig 200, 300 enables the positioning of the second and third screws 60, 70 described above.

The transverse minor body portion 216 includes one, and preferably two fixation screw guides 270, 280 adapted to guide the insertion of guidewire and the drilling of guide holes for the second and third screws 60, 70. The guides 270, 280 are configured to align with the transverse openings through a shank of the first screw 20 fixated bi-cortically and mediolaterally in the metatarsal body fragment and for medial cortical fixation in the bone body fragment and fixation in the metatarsal head fragment.

As illustrated in FIGS. 27, 30, 34, 35, 36, 43 and 44 the two fixation screw guides 270, 280 are comprised of longitudinal openings that are positioned and have respective longitudinal axes that are offset relative to each other. A first one of the screw guides 270 is located laterally and inferiorly (when the jig 200 is stabilised relative to the foot) for guiding the longer one of the second screws 60 for insertion proximally into the metatarsal body fragment. The orientation of the longitudinal axis of the first one of the screw guides 270 facilitates the orientation of the longer one of the second screws 60 so as to pass through the distal one of the transverse apertures 50 of the first screw 20.

A second one of the screw guides 280 is located medially and anteriorly (when the jig 200 is stabilised relative to the foot) relative to the first one of the screw guides 270. The second screw guide 380 is for guiding the shorter one of the second screws 70 for insertion distally into the metatarsal body fragment. The orientation of the longitudinal axis of the second one of the screw guides 280 facilitates the orientation of the shorter one of the second screws 70 so as to pass through the proximal one of the transverse apertures 40 of the first screw 20. Accordingly, the orientation of the longitudinal axes of the transverse apertures 40, 50 in the first screw 20 together with the location and orientation of the first and second screw guide 270, 280 facilitates the proper location and orientation of the second screws 60, 70 as described herein

The method of insertion of the second screws 60, 70 into the metatarsal bone body and head fragments may be via a wire guided technique or technique that does not employ wire guidance. Although not illustrated in the Figures, in a wire guided technique, each of the fixation screw guides 270, 280 is adapted to receive a 1.0 mm guide wire. The guide wires enter the medial cortex of the proximal metatarsal bone body fragment. The guide wires proceed to pass through a respective one of the transverse openings through the shank of the first screw 20 and continue to pass through the lateral cortex of the proximal metatarsal bone body fragment at about 10 mm from the osteotomy. The guide wires continue on to enter the head of the distal metatarsal bone head fragment.

In an embodiment of the method, the guide wires are used to measure the depth to which holes must be drilled. Having measured the required depth, as illustrated in FIG. 35, the fixation screw guides 270, 280 receive a cannulated drill bit guide 290 for guiding a drill bit, such as the bit 295 illustrated in FIG. 39. The bit is preferably a 2.5 mm drill bit including a smaller diameter 1.6 mm section about 7 mm in length from the tip. The drill bit is removed and as illustrated in FIGS. 36 and 37, a driver 298 is employed to drive the second screw 60 and the third screw 70 into position thereby fixing the position of the metatarsal head fragment relative to the proximal body fragment as illustrated in FIG. 38

In another embodiment, guide wires are not employed to measure the required drill depth. Instead the drill bit includes measurement markings to provide an indication that the appropriate depth has been reached.

FIGS. 46 to 51 illustrate another embodiment of the jig 500 that in many respects is identical to the jig 200 of FIGS. 27 to 37 and as such, like reference numerals will be used for like features. The jig 500 of FIGS. 46 to 51 differs from the jig 200 of FIGS. 27 to 37 and the jig 300 of FIGS. 41 to 45 in the form of the jig main body portion 214 and the adjustable support 250.

The jig 500 of FIGS. 46 to 51 includes a body 510 including an elongated main body portion 514 (represented in broken lines in FIGS. 48 to 50 and in solid lines in FIGS. 46, 47 and 51) and a minor body portion 516 extending transversely from a proximal end of the main body portion 514. The main body portion 514 and the minor body portion 516 are slidably coupled together by a releasable coupling 513 comprised of a knurled wheel that is manually rotatable to secure or release the main body portion 514 and the minor body portion 516 relative to each other. When released, the main body portion 514 and the minor body portion 516 are able to slidably move relative to each other in a longitudinal (proximal-distal) direction to lengthen or shorten the jig 500.

The adjustable support 550 of FIGS. 46 to 51 includes a metatarsal head fragment interface 555 that is spaced apart from the body 510 of the jig 500 wherein the distance between the interface 555 and the jig body 510 is adjustable.

The metatarsal head fragment interface 555 of the adjustable support 550 is a substantially saddle shaped member and is supported by a rod 557 that is threadably coupled to a manually rotatable member 558 that is captured by the jig body 510 wherein manual rotation of the rotatable member 558 causes the axial translation of the rod 557 and the interface 555 relative to the jig body 510. The interface 555 includes a linear slot 520 for receiving a complementary shaped head 530 of the rod 557. The head 530 of the rod 557 includes an annular slot 531 spaced a small distance apart from the end of the rod 557 for receiving elongated and opposed flanges 521, 522 arranged either side of the slot 520. The flanges 521, 522 are slidable within the annular slot 531 to thereby allow linear relative movement of the interface 555 and the rod 557. The interface 555 is movably coupled to the rod 557 in a manner that allows the interface 555 to move vertically relative to the rod 557 and jig 500 for positioning the recess 565 to receive the metatarsal.

The manually rotatable member 558 includes and internal thread that threabably engages with an external thread of the rod 557. The manually rotatable member 558 is rotatably coupled to a base member 551 by any suitable means and the base member 551 in turn is mounted to the main body portion 514 of the jig 500. In FIGS. 46 to 51, the manually rotatable member 558 includes a circular flange 559 that is captured within a slot 552 of the base member 551 so as to be freely rotatable therewithin but not permit relative axial movement. Because relative axial movement of the manually rotatable member 558 and the base member 551 are not permitted when the manually rotatable member 558 is rotated the threaded rod 557 is caused to move axially relative to the base member 551 and, in turn, the jig 500.

In the embodiment of FIGS. 46 to 51, the guide aperture 535 for receiving the threaded wire 237 inserted into the medial cortex of the metatarsal body fragment or the medial cortex of the medial cuneiform is positioned outboard or medially of the location of the two fixation screw guides 270, 280. In the illustrated embodiment, this is achieved by locating the guide aperture 535 in an elevated position above the two fixation screw guides 270, 280. This aids in providing clearance between the jig 500 and the medial cortex of the metatarsal body fragment or the medial cortex of the medial cuneiform.

FIGS. 46 and 47 one form of a jig positioning guide member 580 including radio luminescent guides adapted to overlay the proximal metatarsal body fragment and the bone head fragment. The guide member 580 includes a transverse portion 581 coupled at one end to the elongated main body portion 514 of the jig 500. The transverse portion 581 extends at an incline to another end where the transverse portion 581 is coupled to a longitudinal guide portion 582. The guide portion 582 includes a longitudinal guide 583 for aiding in locating the jig relative to the proximal metatarsal body fragment and the bone head fragments and for assisting in accurately translating the bone fragments relative to each other. A transverse guide 584 also aids in positioning the first screw 20.

FIGS. 48 to 51 illustrate another form of the jig positioning guide member 590 including radio luminescent guides adapted to overlay the proximal metatarsal body fragment and the bone head fragment. The guide member 590 includes a transverse portion 591 coupled at one end to a riser 593 which in turn is coupled to the elongated main body portion 514 of the jig 500. The other end of the transverse portion 591 is coupled to a longitudinal guide portion 592 which includes a pair of longitudinal guides 594, 595 for aiding in locating the jig relative to the proximal metatarsal body fragment and the bone head fragments and for assisting in accurately translating the bone fragments relative to each other.

While various embodiments of the invention have been set forth above, it will be apparent to those skilled in the art that various modifications and variations can be made in the present invention without departing from the scope or spirit of the invention. Thus, it is intended that the present invention cover such modifications and variations as fall within the scope of the appended claims and their equivalents.

1. A fixation device for fixing bone head and bone body fragments in an osteotomy procedure, the device including:
a first screw including a shank and an external thread for cortical fixation in the bone body fragment;
the first screw including an opening extending transversely through the shank;
a second screw including a shank and proximal and distal external threads for oblique cortical fixation in the bone body fragment and for cortical fixation in the bone head fragment for relative fixation of the bone body fragment and the bone head fragment; and
the shank of the second screw being locatable within the transverse opening of the first screw.

2. The device of claim 1, wherein the transverse opening comprises a longitudinal passage having a central axis between about 80 degrees and about 100 degrees and preferably about 90 degrees to a central longitudinal axis of the first screw.

3. The device of claim 1, wherein the transverse opening extends linearly through the shank from a first side to a second side and defines a central axis of the transverse opening.

4. The device of claim 1, wherein a second transverse opening extends linearly through the shank from a first side to a second side and defines a central axis of the second transverse opening.

5. The device of claim 1, wherein the external thread of the first screw is a distal external thread.

6. The device of claim 1, wherein the first screw includes proximal and distal external threads.

7. The device of claim 1, wherein a head at the proximal end of the first screw has a proximal face that is part transversely planar at about 90 degrees to the longitudinal axis and part chamfered.

8. The device of claim 7, wherein the chamfered part is at about 30 degrees to the transversely planar part.

9. The device of claim 1, wherein the fixation device is adapted for connecting bone head and body fragments in a distal metatarsal osteotomy procedure and wherein the first screw is adapted for bi-cortical fixation in the proximal body fragment at about 30 degrees to the normal of the longitudinal axis of the proximal body fragment.

10. The device of claim 1, wherein the major diameter of the thread of the first screw is less than or equal to the external diameter of the shank.

11. The device of claim 1, wherein the external diameter of the shank of the second screw and the internal diameter of the transverse aperture are sized for the shank to be locatable in a tight fit within the transverse aperture.

12. (canceled)

13. The device of claim 11, wherein the major diameter of the distal thread of the second screw is less than or equal to the external diameter of the shank and the major diameter of the proximal thread of the second screw is greater than the external diameter of the shank.

14. The device of claim 11, wherein the distal thread of the second screw is adapted for fixation in the lateral cortex of the bone head fragment and the proximal thread of the second screw is adapted for fixation in the medial cortex of the bone body fragment.

15. The device of claim 1, wherein the first screw includes an intermediate external thread between the proximal and distal external threads.

16. The device of claim 1, wherein the fixation device is for connecting first metatarsal bone head and bone body fragments in a hallux valgus osteotomy procedure and wherein the first screw is adapted for distal fixation in the medial cortex and the lateral cortex of the bone body fragment, and the second screw is adapted for proximal oblique fixation in the medial cortex of the bone body fragment and distally traversing the lateral cortex of the bone body fragment and entering the lateral cortex of the bone head fragment for relative fixation of the bone body and bone head fragments, and wherein the shank of the second screw traverses the lateral cortex of the bone body fragment.

17-29. (canceled)

30. A system for fixation of first metatarsal bone head and bone body fragments in a hallux valgus osteotomy procedure, including:
a first screw including a shank and an external thread for cortical and mediolateral fixation in the bone body fragment;
one or more openings extending transversely through the shank of the first screw; and
a jig mountable to the first screw and including one or more guides for guiding one or more second screws to align with the one or more transverse openings through the first screw, wherein the second screw includes a shank and proximal and distal external threads for oblique cortical fixation in the bone body fragment and for cortical fixation in the bone head fragment for relative fixation of the bone body fragment and the bone head fragment.

31. The system of claim 30, wherein at least one of the transverse openings in the first screw comprises a longitudinal passage and at least one of the guides includes a longitudinal passage, wherein the passages are axially aligned when the jig is mounted to the first screw.

32. The system of claim 30, wherein jig includes a pair of the guides that are axially aligned with a respective one of a pair of the transverse openings when the jig is mounted to the first screw and wherein the axes of the pair of guides are angularly displaced from one another.

33-40. (canceled)

41. A surgical method for fixing bone head and bone body fragments in an osteotomy procedure for correcting hallux valgus deformity, the method including:
inserting a first screw mediolaterally in a proximal metatarsal bone body fragment for bi-cortical fixation,
inserting a second screw including a shank and proximal and distal external threads proximally and obliquely through the medial cortex of the proximal metatarsal bone body fragment and through an opening extending transversely through the shank of the first screw and distally traversing the lateral cortex of the bone body fragment and entering the lateral cortex of the bone head fragment for oblique cortical fixation in the bone body fragment and for cortical fixation in the bone head fragment for relative fixation of the body and head fragments.

42. The method of claim 41, including
supporting a jig on the first screw fixated bi-cortically and mediolaterally in the metatarsal body fragment;
stabilising a proximal end of the jig to the proximal metatarsal bone body fragment or medial cuneiform with a threaded wire inserted into the metatarsal body fragment and coupled to the jig;
stabilising a distal end of the jig to the proximal phalanx with a threaded wire inserted into the proximal phalanx and coupled to the jig;
engaging the metatarsal head fragment with an adjustable support mounted to the jig body intermediate the proximal and distal ends of the jig; and
adjusting the lateral translation of the head fragment relative to the body fragment with the adjustable support.

43. The method of claim 41, including inserting a third screw proximally and obliquely through the medial cortex of the proximal metatarsal bone body fragment and through a second transverse opening through the shank of the first screw and entering the bone head fragment for relative fixation of the body and head fragments.

44. The method of claim 43, wherein a guide hole drilled for each of the second and the third screws is guided by the jig through the proximal metatarsal bone body fragment, the transverse openings through the first screw and into the bone head fragment.
